SEQUENCE LISTING (18 sequences)

SEQ ID NO: 1 - DNA, 120 bp, Homo sapiens
tctggattcacctttagcagctatgccatgagctggttccgccaggctccagggaaggagcaggagggggtctcagctattagtggtagtggtggtagcacatactacgcagactccgtg

SEQ ID NO: 2 - DNA, 120 bp, Homo sapiens
tctggattcacctttagcagctatgccatgagctgggtccgccaggctccagggaaggggctggagtgggtctcagctattagtggtagtggtggtagcacatactacgcagactccgtg

SEQ ID NO: 3 - DNA, 40 bp, Artificial Sequence
  PCR primer
ctggttccgccaggctccagggaaggagcaggagggggtc

SEQ ID NO: 4 - DNA, 40 bp, Artificial Sequence
  PCR primer
gaccccctcctgctccttccctggagcctggcggtaccag

SEQ ID NO: 5 - DNA, 120 bp, Homo sapiens
agtttctgaccagggtttctttttgtttgcaggtgtccagtgtgaggtgcagctgcaggagtctgggggaggcttggtacaggctggggggtccctgagactctcctgtgcagcctctgg

SEQ ID NO: 6 - DNA, 120 bp, Homo sapiens
agtttctgaccagggtttctttttgtttgcaggtgtccagtgtgaggtgcagctgttggagtctgggggaggcttggtacagcctggggggtccctgagactctcctgtgcagcctctgg

SEQ ID NO: 7 - DNA, 39 bp, Artificial Sequence
  PCR primer
cagctgcaggagtctgggggaggcttggtacaggctggg

SEQ ID NO: 8 - DNA, 39 bp, Artificial Sequence
  PCR primer
cccagcctgtaccaagcctcccccagactcctgcagctg

SEQ ID NO: 9 - DNA, 33 bp, Artificial Sequence
  Homo sapiens
gtggtcgacgatggaaagatagataccaacatg

SEQ ID NO: 10 - DNA, 32 bp, Artificial Sequence
  PCR primer
gtgctcgagcatctctgtaagcgtcaatctgc

SEQ ID NO: 11 - DNA, 21 bp, Artificial Sequence
  PCR primer
ggtgcagctgcaggagtctgg

SEQ ID NO: 12 - DNA, 21 bp, Artificial Sequence
  PCR primer
ccagactcctgcagctgcacc

SEQ ID NO: 13 - DNA, 21 bp, Artificial Sequence
  PCR primer
cttggtacaggctggggggtc

SEQ ID NO: 14 - DNA, 21 bp, Artificial Sequence
  PCR primer
gaccccccagcctgtaccaag

SEQ ID NO: 15 - DNA, 29 bp, Artificial Sequence
  PCR primer
ggctgagctgggttttccttgttgctatt

SEQ ID NO: 16 - DNA, 29 bp, Artificial Sequence
  PCR primer
ccgccatcctcgccctcctcctggctgtt

SEQ ID NO: 17 - DNA, 29 bp, Artificial Sequence
  PCR primer
cctggagggtcttctgcttgctggctgta

SEQ ID NO: 18 - DNA, 29 bp, Artificial Sequence
  PCR primer
ggctgagctggctttttcttgtggctatt

(19) United States
(12) Patent Application Publication
(10) Pub. No.: US 2009/0271880 A1
(43) Pub. Date: Oct. 29, 2009

(54) BINDING MOLECULES

(76) Inventors: Franklin Gerardus Grosveld, Rotterdam (NL); Richard Wilhelm Janssens, Rotterdam (NL); Dubravka Drabek, Rotterdam (NL)

Correspondence Address: COZEN O'CONNOR, P.C., 1900 MARKET STREET, PHILADELPHIA, PA, 19103-3508, US

(21) Appl. No.: 12/441,581

(22) PCT Filed: Sep. 18, 2007
(86) PCT No.: PCT/IB2007/003647

§ 371 Date: Jun. 17, 2009

(30) Foreign Application Priority Data
Sep. 18, 2006 (GB) 0618345.3

(51) Int. Cl. A01K 67/027; C07K 16/18

(52) U.S. Cl. 800/6; 530/387.3

(57) ABSTRACT

The present invention relates to methods for engineering VH domains to improve their solubility and stability. The invention provides for the incorporation of defined amino acid substitutions based on 3-D structural information into the V segments of a heavy chain locus, expressing the locus in a non-human mammal and selecting soluble VH domains. Further stabilising or solubilising mutations maybe introduced as a result affinity maturation during B-cell maturation in vivo.

### FIELD OF THE INVENTION

The present invention relates to methods for engineering V_{H} domains to improve their solubility and stability. The invention provides for the incorporation of defined amino acid substitutions based on 3-D structural information into the V segments of a heavy chain locus, expressing the locus in a non-human mammal and selecting soluble V_{H} domains as a result of VDJ rearrangement. Further stabilising or solubilising mutations may be introduced as a result affinity maturation during B-cell maturation in video. Such mutations are distinct from those antigen-specific mutations present predominantly in the CDR3 region which optimise antigen recognition and binding.

Heavy chain-only antibodies generated using the methods of the present invention are also described.

In the following description, all amino acid residue position numbers are given according to the numbering system devised by Kabat et al. [1].

### BACKGROUND TO THE INVENTION

### Antibodies

The structure of antibodies is well known in the art. Most natural antibodies are tetrameric, comprising two heavy chains and two light chains. The heavy chains are joined to each other via disulphide bonds between hinge domains located approximately half way along each heavy chain. A light chain is associated with each heavy chain on the N-terminal side of the hinge domain. Each light chain is normally bound to its respective heavy chain by a disulphide bond close to the hinge domain.

When an antibody molecule is correctly folded, each chain folds into a number of distinct globular domains joined by more linear polypeptide sequences. For example, the light chain folds into a variable (V_{L}) and a constant (C_{L}) domain. Heavy chains have a single variable domain V_{H}, a first constant domain (C_{H}1), a hinge domain and two or three further constant domains. The heavy chain constant domains and the hinge domain together form what is generally known as the constant region of an antibody heavy chain. Interaction of the heavy (V_{H}) and light (V_{L}) chain variable domains results in the formation of an antigen binding region (Fv). Interaction of the heavy and light chains is facilitated by the C_{H}1 domain of the heavy chain and the Cκ or Cλ domain of the light chain. Generally, both V_{H} and V_{L} are required for antigen binding, although heavy chain dimers and amino-terminal fragments have been shown to retain activity in the absence of light chain [2].

Within the variable domains of both heavy (V_{H}) and light (V_{L}) chains, some short polypeptide segments show exceptional variability. These segments are termed hypervariable regions or complementarity determining regions (CDRs). The intervening segments are called framework regions (FRs). In each of the V_{H} and V_{L} domains, there are three CDRs (CDR1-CDR3).

Antibody classes differ in their physiological function. For example, IgG plays a dominant role in a mature immune response. IgM is involved in complement fixing and agglutination. IgA is the major class of Ig in secretions—tears, saliva, colostrum, mucus—and thus plays a role in local immunity. The effector functions of natural antibodies are provided by the heavy chain constant region.

In mammals there are five types of antibody: IgA, IgD, IgE, IgG and IgM, with 4 IgG and 2 IgA subtypes present in humans.

| Class | H chain | L chain | Subunits | mg/ml | Notes |
|---|---|---|---|---|---|
| IgG | gamma | kappa or lambda | H₂L₂ | 6-13 | transferred across placenta |
| IgM | mu | kappa or lambda | (H₂L₂)₅ | 0.5-3 | first antibodies to appear after |
| | | | | | immunization |
| IgA | alpha | kappa or lambda | (H₂L₂)₂ | 0.6-3 | much higher concentrations in |
| | | | | | secretions |
| IgD | delta | kappa or lambda | H₂L₂ | <0.14 | function uncertain |
| IgE | epsilon | kappa or lambda | H₂L₂ | <0.0004 | binds to basophils and mast cells |
| | | | | | sensitizing them for certain |
| | | | | | allergic reactions |

IgA can be found in areas containing mucus (e.g. in the gut, in the respiratory tract or in the urinogenital tract) and prevents the colonization of mucosal areas by pathogens. IgD functions mainly as an antigen receptor on B cells. IgE binds to allergens and triggers histamine release from mast cells (the underlying mechanism of allergy) and also provides protection against helminths (worms). IgG (in its four isotypes) provides the majority of antibody-based immunity against invading pathogens. IgM is expressed on the surface of B cells and also in a secreted form with very high affinity for eliminating pathogens in the early stages of B cell mediated immunity (i.e. before there is sufficient IgG to eliminate the pathogens).

Normal B cells contain a heavy chain locus from which the gene encoding a heavy chain is produced by rearrangement. A normal heavy chain locus comprises a plurality of V gene segments, a number of D gene segments and a number of J gene segments. Most of a V_{H} domain is encoded by a V gene segment, but the C terminal end of each V_{H} domain is encoded by a D gene segment and a J gene segment. VDJ rearrangement in B-cells, followed by affinity maturation, provides a rearranged gene encoding each V_{H} domain. Sequence analysis of H₂L₂ tetramers demonstrates that diversity results from a combination of VDJ rearrangement and somatic hypermutation and that diversity in the CDR3 region is sufficient for most antibody specificities [see ref 3].

With the advent of new molecular biology techniques, the presence of heavy chain-only antibody (devoid of light chain) was identified in B-cell proliferative disorders in man (Heavy Chain Disease) and in murine model systems. Analysis of heavy chain disease at the molecular level showed that mutations and deletions at the level of the genome could result in inappropriate expression of the heavy chain C_{H}1 domain, giving rise to the expression of heavy chain-only antibody lacking the ability to bind light chain [4,5].

It has been shown that camelids, as a result of natural gene mutations, produce functional IgG2 and IgG3 heavy chain-only dimers which are unable to bind light chain due to the absence of the C_{H}1 domain, which mediates binding to the light chain [6]. A characterising feature of the camelid heavy chain-only antibody is a particular subset of camelid V_{H} domains, which provides improved solubility relative to human and normal camelid V_{H} domains. The particular subset of camelid V_{H} domains are usually referred to as V_{HH} domains.

It has also been shown that species such as shark produce a heavy chain-only-like binding protein family, probably related to the mammalian T-cell receptor or antibody light chain [7].

The camelid V_{HH} domains found in heavy chain-only antibodies are also characterised by a modified CDR3. This CDR3 is, on average, longer than those found in non-camelid antibodies and is a feature considered to be a major influence on overall antigen affinity and specificity, which compensates for the absence of a V_{L} domain in the camelid heavy chain-only antibody [8,9].

For the production of camelid heavy chain-only antibody, the heavy chain locus in the camelid germline comprises gene segments encoding some or all of the possible heavy chain constant regions. During maturation, a rearranged gene transcript encoding a V_{HH}DJ binding domain is spliced onto the 5′ end of a transcribed gene segment encoding a hinge domain, to provide a rearranged gene encoding a heavy chain which lacks a C_{H}1 domain and is therefore unable to associate with a light chain. Camelid V_{HH} domains contain a number of characteristic amino acids at positions 37, 44, 45 and 47 [see ref 9]. These conserved amino acids are thought to be important for conferring solubility on heavy chain-only antibodies [9]. Only certain camelid V_{H} domains are V_{HH} domains with improved solubility characteristics. In contrast human V_{H} domains derived from display libraries lack these characteristic amino acid changes at the V_{H}/V_{L} interface and consequently are less soluble or “sticky” relative to camelid V_{HH} domains [10]. Unfortunately, the results of efforts to engineer or camelise human V_{H} domains remains unpredictable since the introduction of camelising mutations in the V_{H} domain at the V_{H}/V_{L} interface alone is not sufficient to improve solubility in a predictable manner. It would appear that the introduction of features to enhance solubility may have to be compensated for by as yet undefined mutations elsewhere in the V_{H} domain to maintain structural stability [see review 9].

Heavy chain-only monoclonal antibodies can be recovered from B-cells of camelid spleen by standard cloning technology or from B-cell mRNA by phage or other display technology [10]. Heavy chain-only antibodies derived from camelids are of high affinity. Sequence analysis of mRNA encoding heavy chain-only antibody demonstrates that diversity results primarily from a combination of VDJ rearrangement and somatic hypermutation [11].

An important and common feature of natural camelid and human V_{H} domains derived by phage and other display approaches in vitro is that each domain binds as a monomer with no dependency on dimerisation with a V_{L} domain. These V_{H} binding domains or “nanobodies” appear particularly suited to the production of blocking agents and tissue penetration agents and eliminate the need to derive scFv in vitro when constructing antibody based binding complexes (see PCT/GB2005/002692).

### Production of Antibody-Based Products

The production of antibody-based products by genetic engineering, in particular the production of human or humanised antibody-based products, has resulted in the generation of new classes of medicines, diagnostics and reagents and, in parallel, opportunity for new industry, employment and wealth creation (see www.drugresearcher.com, www.leaddiscovery.co.uk). Antibody-based products are usually derived from natural tetrameric antibodies. There are many patents and applications which relate to the production of antibody-based products. These patents and applications relate to routes of derivation (e.g. from transgenic mice), routes of manufacture and product-specific substances of matter. Such antibody-based products include complete tetrameric antibodies, antibody fragments and single chain Fv (scFv) molecules.

scFv molecules comprise only the variable domains of the heavy (V_{H}) and light (V_{L}) chains linked by a peptide linker to form a single molecule and are usually obtained by screening display libraries (e.g. phage display or emulsion display). Alternatively, they are engineered from natural antibodies by cloning the nucleic acid regions encoding the V_{H} and V_{L} domains into a transcription unit. scFv molecules obviously have a much smaller molecular weight and lack the constant region effector functions of natural antibodies. scFv molecules are often optimized in vitro.

Antibody-based products will represent a high proportion of new medicines launched in the 21st century. Monoclonal antibody therapy is already accepted as a preferred route for the treatment for rheumatoid arthritis and Crohn's disease and there is impressive progress in the treatment of cancer. Antibody-based products are also in development for the treatment of cardiovascular and infectious diseases. Most marketed antibody-based products recognise and bind a single, well-defined epitope on the target ligand (e.g. TNFα).

Manufacture of antibody-based products for therapy remains dependent on mammalian cell culture. The assembly of a tetrameric antibody and subsequent post-translational glycosylation processes preclude the use of bacterial systems, although yeast engineered to produce mammalian glycosylation patterns shows promise as an alternative to mammalian cell-based production systems. Production costs and capital costs for manufacture of antibody-based products by mammalian cell culture are high and threaten to limit the potential of antibody-based therapies in the absence of acceptable alternatives. A variety of transgenic organisms are capable of expressing fully functional antibodies. These include plants, insects, chickens, goats and cattle.

Functional antibody fragments can be manufactured in E. coli but the product generally has low serum stability unless pegylated during the manufacturing process.

Recently, high affinity V_{H} domains have been selected from randomised human V_{H} domains in display libraries derived from heavy chain-only antibody produced naturally from antigen challenge of camelids or derived from V_{H} domain libraries made from camelids. These high affinity V_{H} domains have been incorporated into antibody-based products. These V_{H} domains, also called V_{HH} domains, display a number of differences from classical V_{H} domains, in particular a number of mutations that ensure improved solubility of the heavy chains in the absence of light chains. Most prominent amongst these changes is the presence of charged amino acids at positions 44, 45 and 47. It is supposed that these changes compensate for the absence of V_{L} through the replacement of hydrophobic residues by more hydrophilic amino acids, thereby maintaining solubility in the absence of the V_{H}/V_{L} interaction [for review see ref 9 and other references cited therein].

A number of groups have worked on the generation of heavy chain-only antibodies derived from natural tetrameric antibodies. Jaton et al. [2 and other references cited therein] describe the separation of the reduced heavy chain components of an affinity purified, well-characterised rabbit antibody, followed by the subsequent renaturation of the individual heavy chains. Immunological characterisation of the renatured heavy chains demonstrated that a heavy chain homodimer alone, free of light chain, binds antigen.

Later, Ward et al. [10] demonstrated unambiguously that cloned murine V_{H} regions, when expressed as soluble protein monomers in an E. coli expression system, retain the ability to bind antigen with high affinity. Ward et al. [10] describe the isolation and characterisation of V_{H} domains and set out the potential commercial advantages of this approach when compared with classic monoclonal antibody production (see last paragraph). They also recognise that V_{H} domains isolated from heavy chains which normally associate with a light chain lack the solubility of the natural tetrameric antibodies. Hence Ward et al. [10] used the term “sticky” to describe these molecules and proposed that this “stickiness” can be addressed through the design of V_{H} domains with improved solubility properties.

The improvement of V_{H} solubility has subsequently been addressed using combinations of randomized and site-directed approaches using phage display. For example, Davies and Riechmann [12] and others (see WO92/01047) incorporated some of the features of V_{H} domains from camelid heavy chain-only antibodies in combination with phage display to improve solubility whilst maintaining binding specificity.

Human V_{H} domains may be engineered in vitro for improved solubility characteristics [9, 12]. Where V_{H} binding domains have been derived from phage libraries, intrinsic affinities for antigen remain in the low micromolar to high nanomolar range, in spite of the application of affinity improvement strategies involving, for example, affinity hot spot randomisation [1,3]. However, the engineering of mammalian V_{H} domains to improved solubility remains unpredictable. Moreover, it is apparent that, in spite of published reports (12 14, 15), the introduction of “camelising” mutations is insufficient to provide predictable outcomes and further mutations are required if enhanced solubility is to be obtained in the absence of aggregation [9].

Human V_{H} or camelid V_{HH} domains produced in vivo, unlike V_{H} produced by phage display technology, have the advantage of improved characteristics in the CDR3 region of the normal antibody binding site as a result of somatic mutations introduced as a result of affinity maturation, in addition to diversity provided by D and J gene segment recombination. Camelid V_{HH}, whilst showing benefits in solubility relative to human V_{H}, is antigenic in man and must be generated by immunisation of camelids or by phage display technology.

Recently, methods for the production of heavy chain-only antibodies in transgenic non-human mammals have been developed (see WO02/085945, WO02/085944; and [1,6]). Functional, high affinity, heavy chain-only antibody of potentially any class (IgM, IgG, IgD, IgA or IgE) and derived from any mammal can be produced using transgenic non-human mammals (preferably rodents) as a result of antigen challenge.

These soluble heavy chain-only antibodies were derived from an antibody heavy chain locus in a germline (i.e. non-rearranged) configuration that contained two llama V_{HH} (class 3) gene segments coupled to all of the human D and J gene segments and gene segments encoding all the human constant regions. The gene segments encoding each of the constant regions had a deletion of the C_{H}1 domain to prevent the binding of light chain. In addition, the locus contained the antibody LCR at the 3′ end and other intragenic enhancer elements to ensure a high level of expression in cells of the B lineage [17].

On challenge with antigen, VDJ recombination and B-cell activation with associated affinity maturation as a result of somatic mutations was observed. Somatic mutations were observed in the llama V gene segment in addition to the expected mutations predominantly in the CDR3 region [16].

It seems likely that the optimal production and selection of heavy chain-only antibodies comprising high affinity, soluble V_{H} binding domains (whether of human, camelid or other origin) will benefit from alternative approaches to those dependent on selection from randomised phage libraries which do not facilitate in; vivo recombination and affinity maturation.

There remains a need in the art to maximise heavy chain-only antibody diversity and B-cell response in vivo and, in particular, to generate a functional repertoire of class-specific, soluble, human heavy chain-only antibodies and functional V_{H} heavy chain-only binding domains which retain maximum antigen-binding potential in the absence of aggregation for use in diverse clinical, industrial and research applications.

Therefore, there remains a need in the art to produce V gene segments which, when recombined with D and J segments in transgenic non-human mammals in response to antigen challenge, generate functional, soluble, antigen-specific, heavy chain-only antibodies in the absence of aggregation (stickiness).

### THE INVENTION

The present inventors have surprisingly overcome the limitations of the prior art and shown that soluble, fully-human V_{H} domains can be derived by the incorporation of human V segments into a heavy chain locus wherein the V gene segments (i) have been modified at the V_{L} interface so as to reduce hydrophobicity and (ii) have additional mutations introduced to overcome structural instability. Such additional mutations may be beneficial independent of those mad at the V_{L} interface. The selection of functional V segments occurs in transgenic mice. Further improvements may be incorporated in vivo into the V_{H} domain by natural selection as a result of B-cell dependent affinity maturation following a response to antigen Therefore, the present invention provides a method for producing a heavy chain-only antibody comprising:

challenging with an antigen a non-human mammal having a heavy chain locus which:
comprises a plurality of V gene segments at least one of which of which encodes one or more amino acid mutations (i) at the V_{L} interface so as to reduce hydrophobicity and (ii) at other positions so as to overcome structural instability or decrease hydrophobicity;
comprises at least one D gene segment and at least one J gene segment;
does not contain any gene segments encoding a C_{H}1 domain; and
when expressed in response to antigen challenge, produces a heavy chain-only antibody having a soluble V_{H} domain encoded by a V_{H} gene which includes a, preferred V gene segment incorporated as a result of VDJ rearrangement into said V_{H} gene.

Preferably the heavy chain locus comprises a plurality of V gene segments a plurality of which encode-one or more amino acid mutations as described at (i) and (ii) abover

The non-human mammal may be produced by: producing in vitro a transgene including said-locus; and introducing said transgene into a suitable cell from which said non-human mammal is to be produced. The cell may be an embryonic stem cell or an ocyte.

Alternatively, said non-human mammal is produced by homologous recombination in which said plurality of mutated V gene segments, and, optionally, said D and J gene segments, replace the equivalent gene segments in an endogenous heavy chain locus in the non-human mammal.

Where only one locus is present, preferably all the V gene segments are engineered.

Preferably, the non-human mammal includes multiple heavy chain loci at least one of which is as defined above and each of which is on a different chromosome. If desired, the loci on each chromosome may be the same. Alternatively, the loci on each chromosome may be different. Where the loci are different, they may comprise combinations of natural and engineered V gene segments. Alternatively, some may comprise natural V gene segments and some engineered V gene segments.

Preferably, the V gene segments are mutated human V gene segments.

Preferably, the locus includes multiple D gene segments.

Preferably, the locus includes multiple J gene segments.

If desired, the locus may contain at least one gene segment encoding a constant region.

Preferably, the locus contains multiple gene segments encoding a constant region.

Preferably, each gene segment encoding a constant region is human.

Preferably, each V, D and J gene segment is human.

Preferably, each V gene segment encodes a protein which has a mutation at one of positions 37, 44, 45 and 47. More preferably, each V gene segment encodes a protein which has mutations at all of positions 37, 44, 45 and 47.

Preferably, the V gene segment encodes a protein wherein at position 37, the residue is phenylalanine (F), at position 44, the residue is glutamic acid (E), at position 45, the residue is glutamine (Q) and/or at position 47, the residue is glycine (G).

Preferably, each V gene segment encodes a protein which has a mutation at either of positions 5 and 14. More preferably, each V gene segment encodes a protein which has mutations at both of positions 5 and 14.

Preferably, the V gene segment encodes a protein wherein at position 5, the residue is glutamine (Q) and/or at position 14, the residue is alanine (A).

A “V_{H} domain” in the context of the present invention refers to an expression product of a V gene segment when recombined with a D gene segment and a J gene segment. Preferably, the V_{H} domain as used herein remains in solution and is active in a physiological medium and at physiological temperature in mammals without the need for any other factor to maintain solubility. Optionally, the solubility and stability of the V_{H} domain maybe improved by somatic mutation following VDJ recombination. There is no evidence for the presence of the enlarged CDR3 loop present in V_{HH} domains but not in V_{H} domains produced by the camelid species. The V_{H} domain is able to bind antigen as a monomer and, when expressed with an effector constant region, may be produced in mono-specific, bi-specific, multi-specific, bi-valent or multivalent forms, dependent on the choice and engineering of the effector molecules used (e.g. IgG, IgA IgM etc.) or alternative mechanisms of dimerisation and multimerisation. Any likelihood of binding with a V_{L} domain when expressed as part of a soluble heavy chain-only antibody complex has been eliminated due to the absence of a CH1 domain [16].

The properties of the V_{H} domain may be altered or improved by selecting or engineering V, D and/or J gene segments which encode sequences with the required characteristics. Preferably, the V_{H} domain will have improved solubility. Preferred methods of improving solubility of a V_{H} domain incorporate rational design based on known 3-D structure [18] followed by incorporation of engineered V segments into a heavy chain locus, allowing the expression, affinity maturation and selection of soluble heavy chain-only antibodies from activated B-cells in a non-human mammal of choice. Preferred D and J segments, whether natural or engineered, may also be incorporated into the locus.

The method of the present invention provides an ideal tool for selecting V gene segments which are capable of producing soluble V_{H} domains. Using the method of the present invention, a V_{H} heavy chain-only antibody will only be produced if the V_{H} domain, translated from the somatically mutated, recombined V, D and J gene segments, is soluble. B cells failing to produce soluble antibodies will not survive, whilst those which do will undergo further natural selection in vivo through affinity maturation and the incorporation of favourable mutations in the V_{H} gene following VDJ rearrangement. The resulting antibodies will be both soluble and show high antigen specificity. Therefore, the method allows the selection of mutated V_{H} domains, more preferably mutated human V_{H} domains, which are soluble. Since it is evident that several synergistic mutations may be necessary to achieve this result, and that these may differ dependent on the V gene segment expressed, preferred mutations are introduced into selected V segments prior to their incorporation into a heavy chain locus. The incorporation of other beneficial mutations and selection may then occurs as a result of affinity maturation in vivo. Alternatively, an engineered V gene segment introduced into the locus may encode part of a V_{H} domain which shows solubility and stability in the absence of further affinity maturation. Thus, affinity maturation will contribute to antigen binding specificity and affinity alone.

These soluble V_{H} domains can be analysed by sequencing the V_{H} domains found in B cells producing soluble, high affinity antibodies. This will allow the identification of further somatic mutations in the V gene segment which impart increased solubility. Once identified, these mutations can be incorporated into new V gene segments. These can again be incorporated into a heavy chain locus, which can then be expressed in further transgenic non-human mammals and the process of selecting soluble V_{H} domains repeated. Beneficial somatic mutations in the D and J segments can also be identified.

Thus, the invention firstly utilises information deduced from the crystal structures of camelid V_{HH} and classical V_{H} regions and information from known soluble V_{H} domains found in nature and secondly uses transgenic non-human mammals to select for soluble V_{H} domains resulting from VDJ rearrangement in B-cells following antigen stimulation. Preferred engineered V gene segments can then be used to generate a further transgenic non-human mammal carrying a heavy chain locus which incorporates V gene segments which provide V_{H} domains with superior solubility characteristics as a result of antigen challenge.

Here we describe the generation of completely human, heavy chain-only antibodies in transgenic nonhuman mammals. The main problem in generating such antibodies is the low solubility of non-camelid (e.g. human, rabbit, mouse) V_{H} domains in the absence of interaction with V_{L}. The V_{H} domain has a large hydrophobic surface that normally interacts with a similar region of the light chain variable domain (V_{L}) in normal tetrameric antibodies, providing a soluble complex. However, when dissociated from V_{L}, this hydrophobic region is responsible for solubility problems sometimes encountered in isolated V_{H} domains.

The present invention provides for the introduction of one of more amino acid substitutions in camelid and non-camelid V_{H} domains, in particular via the V gene segment encoding part of the human V_{H} domain, so as to overcome the solubility problem whilst maintaining or strengthening the three dimensional structure of the mutated VH domain. Mutated V gene segments are then incorporated in a locus containing the D, S and constant region gene segments. The locus may include any necessary intragenic regulatory elements, and the Ig LCR as described previously [16]. Such a locus, or preferably such loci as described above, are then introduced into a non-human mammal, such as a mouse, for instance by microinjection or any other suitable technique. Transgenic non-human mammals carrying this locus respond to antigen challenge, resulting in rearrangement of the locus in cells of the B lineage and the production of soluble heavy chain-only antibodies as a result of immunization and maturation in vivo.

Thus, there is provided a method for the production of mutations in non-camelid, in particular human, V_{H} domains so as to overcome the solubility problem by producing a non-camelid V_{H} heavy chain locus containing non-camelid engineered V gene segments as well as D, J, and, optionally, C gene segments and regulatory elements such as Ig enhancers and the Ig LCR, introducing such a locus into a non-human mammal and challenging the transgenic mammal carrying this locus with antigen, resulting in rearangement of the locus in cells of the B lineage and the production of soluble heavy chain-only antibodies as a result of immunization and maturation in vivo.

Preferably, the engineered V gene segment encodes one or more amino acid substitutions at the V_{L} interface and additional amino acid substitutions in order to maintain three dimensional structural stability. Such additional mutations may also be beneficial independent of any other mutation by increasing hydrophilicity

By way of example we describe mutations introduced into 8 human subfamily 3 V_{H} regions, one subfamily 1 V_{H} region and 1 subfamily 5 V_{H} region (see FIG. 1) as a result of structural interactions based on 3D knowledge of the camelid, V_{HH} and, human V_{H} domains (www.rcsb.org/pdbl). Family 3 is preferred because its V_{H} domains have similarities with camelid V_{HH} domains, but the approach can be used to generate improved solubility characteristics in any V_{H} domain (FIG. 1).

In particular, two mutations in the human sequence are made based on the data obtained with camelid V_{HH} regions, in particular at two positions: a phenyalanine (F) at position 37, replacing an isoleucine (I) or valine (V); and a glutamic acid (E) at position 44 replacing a glycine (G).

In camelid sequences, position 45 is a charged amino acid. The leucine 45 in the human sequence is not substituted with a charged amino acid but with a glutamine (Q, i.e. different from camelid), because it establishes a hydrogen bridge with the carbonyl group of glycine 116, establishing a structure similar; to that observed in camelid V_{HH} around 116 and provide stacking. In addition, a glycine (G) substitutes the tryptophan (W) or tyrosine (Y) at position 47 to establish an interaction with the tyrosine (Y) at position 58.

These four amino acid changes are introduced in all of the human V_{H} sequences shown in FIG. 3.

In addition, two other substitutions are introduced either together in the V_{H} sequences 3-11, 3-23 and 3-53 or individually in the 3-23 V_{H} sequence only. At position 5, a glutamine (Q) is introduced for a valine (V) or leucine (L) to increase the surface hydrophilicity of the V_{H} region. The proline (P) at position 14 is substituted with an alanine (A) to establish an interaction with amino acid 127 in the DJ region and reinforce the structure of the V_{H} domain. Such interaction does not take place with the normal P at position 14.

The mutations made in the V gene segments are designed to maintain a balance between increased solubility and decreased overall stability of the V_{H} domain through increased surface hydrophilicity. The present invention also contemplates the use of any other mutations which will achieve the required balance of improved solubility whilst maintaining three dimensional structural stability.

The V gene segments are incorporated head to tail into the previously-described locus [16], combining the engineered human V segments with, preferably, the human D, J and optional constant region gene segments. In this example, seventeen engineered human V segments (FIG. 3) are incorporated with human D and J segments into one locus containing the Cγ heavy chain constant region lacking the C_{H}1 region. Alternatively several loci may be introduced into the mice each containing a different subset of the engineered V segments with the same or different constant regions. Due to allelic exclusion (see PCT/IB2007/001491), only one of these loci will be chosen as the productive locus in vivo.

Preferably, the transgenic, non-human mammals are immunised with a broad spectrum of antigens to produce a variety of heavy chain-only antibodies (although antibody production will inevitably occur due to exposure to environmental antigens). The V_{H} domain sequences of the antibodies thus produced are then compared with one another to identify common mutations. Such common mutations are most likely to be the ones which improve solubility. V_{H} domains derived from such antibodies show solubility and stability under physiological conditions and lack the “sticky” nature of VH domains isolated from phage and alternative display libraries. Optionally, the VH domains comprise only human sequences and so lack the antigenicity of the camelid-derived V_{H} domains when used as therapeutics in man. Advantageously, these mutations are built into new loci for the production of stable, soluble heavy chain-only antibodies following challenge with specific antigens.

The transgenic non-human mammal is preferably a rodent such as a rabbit, guinea pig, rat or mouse. Mice are especially preferred. Alternative mammals such as goats, sheep, cats, dogs or other animals may also be employed. Preferably, the mammal is a mouse.

Preferably, transgenic non-human animals are generated using established oocyte injection technology and, where established, embryonic stem (ES) cell technology or cloning.

Alternatively, a locus encoding heavy chain-only antibodies (devoid of C_{H}1 regions) could be introduced into mice by replacement of the endogenous mouse heavy chain locus through homologous recombination. This could be achieved in several ways known to a person skilled in the art.

For example, using homologous recombination, one could insert lox or fit recombination sites at each end of the locus using standard recombination technology in ES cells (e.g. see www.ncrr.nih.gov/newspub/KOMP_Lloyd_{—}1-18-2007.ppt). After treating the recombined ES cells with cre (acting on lox sites) or flp (acting on frt sites) recombinase, respectively, the entire murine locus would be removed.

In order to introduce the human sequences in place of the mouse locus, one would introduce the 5′ end sequences of the mouse locus at the 5′ end of the human locus and the 3′ end of the mouse locus at the 3′ end of the human locus. This is most efficiently done by the recombineering of BACs or PACs containing the normal or engineered human locus. This new locus containing the mouse sequences at either end is recombined via these mouse sequences into the position where the mouse locus was in the recombined ES cells. As a result, the murine locus is replaced by the human locus.

A number of variations are possible in the above scheme. The homologous recombination of very large loci is not efficient and hence the engineering may be done in a number of smaller steps, each time replacing parts of the locus with a new human part. Instead of BACs or PACs, YACs and recombination in yeast could be used to introduce the lox sites into the human locus. The recombination could be done at different positions of the murine locus, for example the human locus may not contain the human LCR when the 3′ lox site is introduced to the 5′ side of the murine LCR. As a result, expression of the human locus would be driven by the murine LCR. A number of variants on the recombination procedures are possible in the ES cells and the construction of the BACs could make use of recombineering techniques such as Gateway™ cloning (InVitrogen). An example of this approach is the Regeneron VelocImmune transgenic mouse (www.Regeneron.com).

Alternatively, the recombination could be carried out in a murine somatic cell rather than ES cells. After recombination and replacement of the murine by human loci, the nucleus of the recombined cell could be used to generate mice using nuclear transfer cloning using standard procedures known to the person skilled in the art (http://www.liebertonline.com/toc/clo/9/1). Obviously, the host somatic cells could be of any mammalian origin and be used to delete its immunoglobulin locus and use the nuclei for a nuclear transfer-mediated cloning of that mammal.

In all of these procedures, the locus to be recombined and replace the endogenous locus could be from a mammal other than human to produce antibodies of that particular species. A number of variations are possible in the above scheme using homologous recombination for locus recombineering, particularly in host species lacking ES cell technology.

The generation of transgenic mice comprising heavy and light chain loci in a mouse background where the V, D and J regions of the murine heavy chain and the V and J regions of the murine light chain loci have been engineered such that these regions comprise the equivalent human gene segments or sequences is known (see EP1399575 and www.Regeneron.com). Whilst a painstaking approach, the identical strategy maybe used to generate a functional heavy chain locus in transgenic mammals. Thus, the host heavy chain loci are selectively engineered so that natural or engineered V gene segments of the species of choice replace host heavy chain V segments. Host D and J segments are similarly replaced and the host heavy chain constant regions are either replaced by constant regions of choice (devoid of C_{H}1) or the C_{H}1 domains are deleted from the host heavy chain loci. Preferably, the inserted sequences of choice are human gene sequences, optionally engineered to optimize the physical characteristics of the V_{H} domains derived subsequently in response to antigen challenge. The advantage of this approach is that host regulatory elements residing outside of the coding sequences are retained, so maximizing the likelihood of normal molecular and cellular function in vivo in response to antigen challenge. Advantageously, the host is a rodent, preferably a rat or mouse, allowing the application of standard laboratory molecular and cellular techniques for characterization of the resultant antibodies. The host may potentially be any mammal for example sheep, pig, cow, goat, rabbit, horse, cat or dog. Advantageously, but not essentially, antibody heavy and, optionally, light chain loci endogenous to the mammal are deleted or silenced when a heavy chain-only antibody is expressed according to the methods of the invention. Operationally, heavy chain antibody loci are introduced into a wild type, preferably mouse, background and then crossed into a background where endogenous genes are deleted or silenced so that only transgenic heavy chain loci respond to antigen challenge in the second generation, leading to B-cell activation and circulating heavy chain-only antibodies in blood.

The methods of generating heavy chain-only antibodies as described above may be of particular use in the generation of antibodies for human therapeutic use, as often the administration of antibodies to a species of vertebrate which is of different origin from the source of the antibodies results in the onset of an immune response against those administered antibodies. The antibodies produced by the method of the invention have the advantage over those of the prior art in that they are of substantially a single or known class and preferably of human origin.

Accordingly, a further aspect of the invention provides a transgenic non-human mammal comprising one or more heterologous V_{H} heavy chain loci as defined above. The transgenic non-human mammal may be engineered to have a reduced capacity to produce antibodies that include light chains.

Antibody-producing cells may be derived from transgenic non-human mammals as defined herein and used, for example, in the preparation of hybridomas for the production of heavy chain-only antibodies as herein defined. In addition or alternatively, nucleic acid sequences may be isolated from these transgenic non-human mammals and used to produce V_{H} domain heavy chain-only chain antibodies or bi-specific/bi-functional complexes thereof, using recombinant DNA techniques which are familiar to those skilled in the art.

Alternatively or in addition, antigen-specific heavy chain-only antibodies may be generated by immunisation of a transgenic non-human mammal as defined herein.

Accordingly, the invention also provides a method for the production of heavy chain-only antibodies in response to antigen challenge of a transgenic non-human mammal as defined above. This may be a direct response to an environmental antigen (eg pathogen) or as a result of immunisation with a target antigen. Antibodies and fragments thereof may be may be isolated, characterised and manufactured using well-established methods known to those skilled in the art. These antibodies are of particularly use in the methods described in PCT/GB2005/00292.

The invention is now described, by way of example only, in the following detailed description which refers to the following figures.

### FIGURES

FIG. 1: A three dimensional model of a V_{H} domain showing the positions of the mutations. The crystal structure is obtained from the PDB public database (see text). The position of the engineered amino acid changes are shown from normal>engineered, the arrow indicates the position of the change in the three dimensional structure. The linear sequences of the engineered V_{H} segments are shown in FIG. 3.

FIG. 2: PCR based strategy to introduce desired amino acid changes into human V_{H} domains. This scheme shows the basic strategy to introduce the desired mutations by a first round PCR using primers from the 5′ and 3′ end to amplify the desired VH segment (top line, primers 1 and 2). The 5′ half and 3′ half are then amplified separately using overlapping primers containing the mutations (indicated by a star in primers 3 and 4, second line). Next, the mutated 5′ and 3′ halves are mixed, denatured and renatured (line 3). Primers 1 and 2 are added and the entire V gene segment is amplified containing the mutations (line 4). The strategy is repeated with other primers if more mutations are to be generated (see text).

FIG. 3: A human heavy chain only locus containing 17 mutated V_{H} segments. The numbers preceding the V gene segment sequences in the upper part of the panel indicate their order as shown by the numbers in the bottom part of the panel. The light grey shading indicate amino acid changes that have been generated in all of the V gene segments by the amino acid indicated above the shading (position 37: F; position 44: E; position 45: Q; and position 47: G). The dark grey shaded amino acids are replaced by the amino acid indicated above the dark grey shaded amino acids (position 5: Q; position 14: A). These latter amino acids were generated in V gene segments that were first modified at the central light grey positions.

FIG. 4: Southern blot hybridised of 8 founder transgenic lines (A-H) containing the 17V_{H} regions. The Southern blot was hybridised with a V_{H}23 probe. C-wt is a non-transgenic mouse control and MDS is a transgenic mouse line control reported by in [16].

FIG. 5 Western blot of serum of the 17V_{H} transgenic mouse line A. Western blot showing the expression of human heavy chain-only antibody (HAb) in transgenic mice serum containing the 17V_{H} locus under reducing conditions (i.e. showing single rather than dimer chains although the dimers are visible). Marker lane shows molecular weight bands, the lane human serum contains normal human IgG. Molecular weights are indicated, the HAb has the predicted size of approx. 45 Kd.

FIG. 6: Sequences of the 5′ end of the different V_{H} regions. The sequences at the 5′ end of the different V_{H} regions used in the 17V_{H} construct. Mismatches with the consensus V_{H}3 sequence are indicated by white shading.

FIG. 7: PCR products of the cDNA prepared from the transgenic mice carrying a locus with 17V_{H}. The cDNA was amplified using either the V_{H}all primer or a combination of all forward primers (see text above) and both reverse primers for cγ2 and Cγ3 (see text). The forward primer combination used is indicated above the lanes. The size markers in the marker lane are shown on the left. The numbers in the bottom of the lanes indicate relative quantity of cDNA in the PCR reaction

FIG. 8: Example of gel electrophoresis of inserts derived from the 17V_{H} transgenic mice. Plasmid DNA minipreps were prepared by standard methods and cleaved by EcoRI. The 12 preps on the left are derived from the cDNA synthesis and amplification with the V_{H}3 all forward primer, the 12 DNA preps on the right with the V_{H}3 all plus the other forward primers (see text. The size of the relevant marker bands is indicated on the left.

FIG. 9. Examples of engineered VH domains comprising mutated human VH segments and natural D and J segments. Examples of sequenced inserts from FIG. 8, showing that the different engineered subclass VH regions and engineered different VH regions within a subclass are used in productive VDJ rearrangements. The result also shows that use J4 is used most commonly as in human. As expected diversity is primarily created by the VDJ rearrangement.

### EXAMPLES

The work described in the following examples is based on the work described in [16]. It is therefore necessary to read [16] in order to understand fully the following examples. The disclosure of [16] is incorporated herein fully by reference.

### Example 1

The construction of transgenic non-human mice containing functional heavy chain-only gene loci.

In a preferred embodiment of the first aspect of the invention, a number of human V gene segments are cloned onto a multiply-modified human locus containing the entire D region, the entire J region, the Cμ, Cγ2, Cγ3 and Cα regions and the 3′LCR using those methods described in [16] and known in the art.

All human V gene segments are available on a yeast artificial chromosome (YAC). The functional human V gene segments are cloned in sets onto the locus described in [16], i.e. comprising the human D plus J and Cμ, Cγ2, Cγ3 each lacking a C_{H}1 plus 3′ LCR. The Cα region plus switch regions may be cloned with lox sites (the C_{H}1 would be removed by homologous recombination).

The functional V gene segments may be cloned together, with any multiple on each locus. Initially, the functional human V gene segments will each be cloned. To each of these initial constructs, a second gene will be added by conventional methodology (e.g. using XhoI-SalI restriction digestion/ligation, ligation of XhoI and SalI compatible sites destroys both).

A second round of cloning may be carried out in which the genes of every second clone will be added to those of the preceding clone, e.g. the two genes from clone 2 will be added to the 2 genes from clone 1, the two genes from clone 4 will be added to the two genes from clone 3, and so on. The second round of cloning will result in 9 clones of 4 genes etc.

The above process may be terminated at any point to achieve the desired number of V gene segments. The D, J and constant regions will be added to these V gene segments. These final loci can then be introduced into transgenic mice as described in [16].

A similar strategy maybe used to incorporate diverse natural V gene segment such as those derived from the human T-cell receptor family or immunoglobulin light chains. The origin of material need not be limited to human but maybe derived from any source including mammals and shark, providing that VDJ rearrangement occurs with associated affinity maturation in response to antigen challenge in a B-cell specific manner. Other routes to construct functional heavy chain-only loci comprising the genes and regulatory elements described in [16] will be familiar to those skilled in the art (e.g. replacement of host gene sequences by homologous recombination). Preferred non-human hosts are rodents, especially mice, but those skilled in the art of, for example, transgenic pigs, cattle, sheep and goats will appreciate that such gene constructs described above can be readily adapted, incorporated and expressed in the chosen host genome.

There is no need to provide non-human mammalian hosts lacking functional endogenous immunoglobulin genes. Selected transgenic non-human mammals comprising functional heavy chain gene loci may be crossed at a later stage with non-human mammals lacking functional endogenous immunoglobulin gene expression to produce transgenic non-human mammals with functional heavy chain loci which secrete only functional heavy chain-only antibody into the plasma in a B-cell dependent manner.

### Example 2

Generation of a functional human heavy chain locus comprising 17 engineered V gene segments and the production of soluble heavy chain-only antibody in transgenic mice.

Antibody loci are generated by “cassetting” mutated human V gene segments into a locus containing all of the human D regions, all of the human J regions, any one (or more) of the human constant regions from which the C_{H}1 domain has been removed and the immunoglobulin LCR. The removal of the C_{H}1 domain ensures that expression of the locus will result in the production of heavy chain only immunoglobulins ([16] and the example above). Inclusion of the LCR in addition to intragenic enhancer elements maximizes the B-cell specific gene regulation and overcomes position effects due to the random nature of integration of the immunoglobulin heavy chain loci in the host genome.

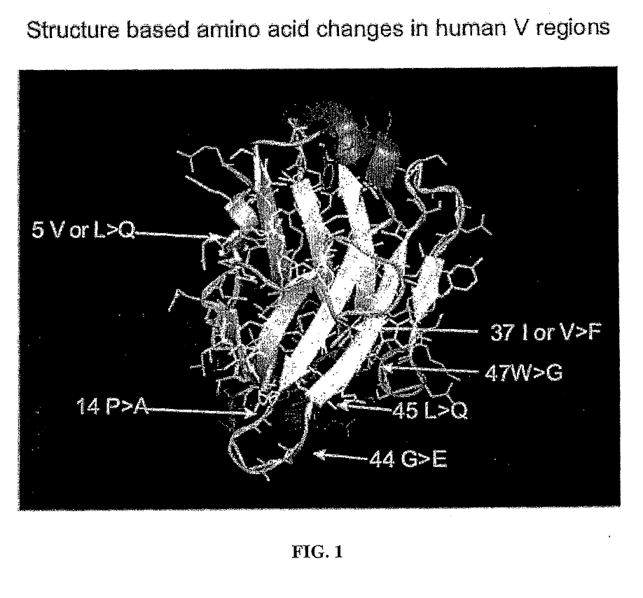

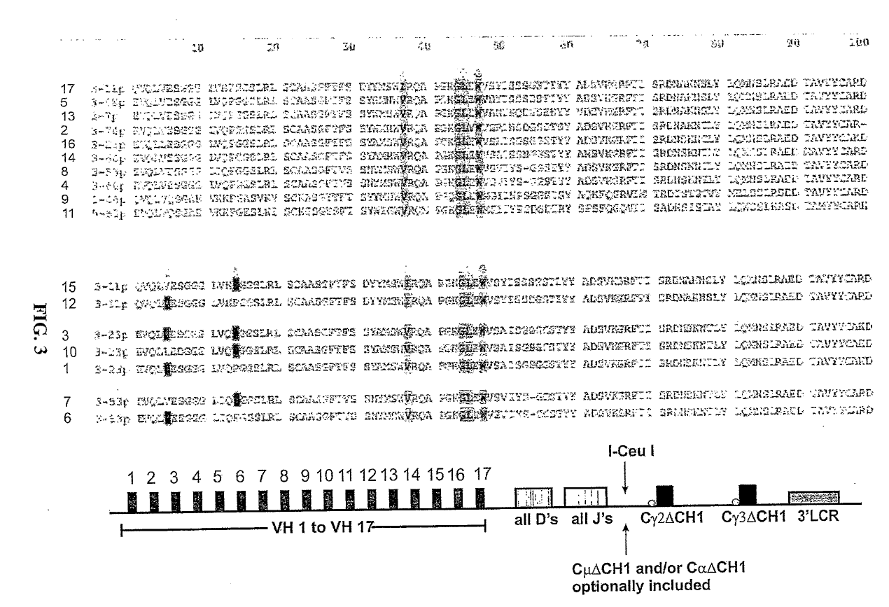

This example describes the generation of a completely human locus containing 17 V gene segments (FIG. 1 bottom). This locus is as described in [16] with exception that the 2 V_{HH} gene segments of llama origin have been replaced by 1.7 human V gene segments of differing subclasses containing defined mutations. The choice of V gene segment is not limiting. However, in this example, subclass 3 V gene segments have been chosen because these are most like V_{HH} gene segments and are expected to produce V_{H} domains which are more soluble than the other subclasses. The next most soluble V_{H} domains are thought to be subclasses 1 and 5 which have also been included. Next, a number of mutations are introduced into the cloned V gene segments by routine PCR-based procedures, where the desired nucleotide mutations are built into the primers, to generate a number of mutations which, based on structural analysis, would be predicted to increase the solubility while maintaining the stability of the VH regions. The preferred mutations are derived from a study of the crystal structures (FIG. 1) and primary amino acid sequences from public databases (in particular in the PDB public database structures 1DEE, human, and 1QDO, llama The amino acid changes are (see FIGS. 1 and 3):
a change from I or V to F at position 37, which is a favourable mutation independent of the other mutations;
a change from G to E at position 44;
a change from L to Q at position 45; and
a change from W to G at position 47.

The latter three mutations are made in combination and add charge (solubility) and improve π-stacking (stability) in a crucial part of the V_{H} domain.

A change is also made from P to A at position 14, which would be favourable independent of the other mutations as it provides an interaction with the DJ region of the molecule (position 127 in the crystal structures in the PDB public database).

A change is also made from V or L to Q at position 5, which is favourable independent of the other mutations as it improves surface hydrophilicity

The individual V gene segments were first mutated to introduce the F, E, Q and G changes in the central part of the V gene segments (FIG. 3 top). Next the VH3-23p, 3-11p and 3-53p regions were further mutated to introduce either the change to A at position 14 or the change to Q at position 5 or a combination of both as shown in FIG. 3.

The individual V gene segments were all cloned between SalI and XhoI sites which allows the generation of cassettes. Every time two V regions are ligated together a SalI and XhoI are ligated together destroying both sites. As a result, the 2 V gene segments again have one SalI and one XhoI site, allowing another round of cassetting etc. In the case of the 17 V gene segments, they were cloned as cassettes of four, four, four, three and two to arrive at the final seventeen. The cassetting was done into a SalI site cloned between two PspI sites. The final 17 V gene segments were isolated as a PspI fragment and cloned into the PsPI site of a PAC containing the D, J and Cγ2 and Cγ3 and LCR regions (see [16] with the exception that the locus does not contain lox sites). The PspI site was introduced into the PAC by routine manipulation.

### Production of Engineered V Gene Segments

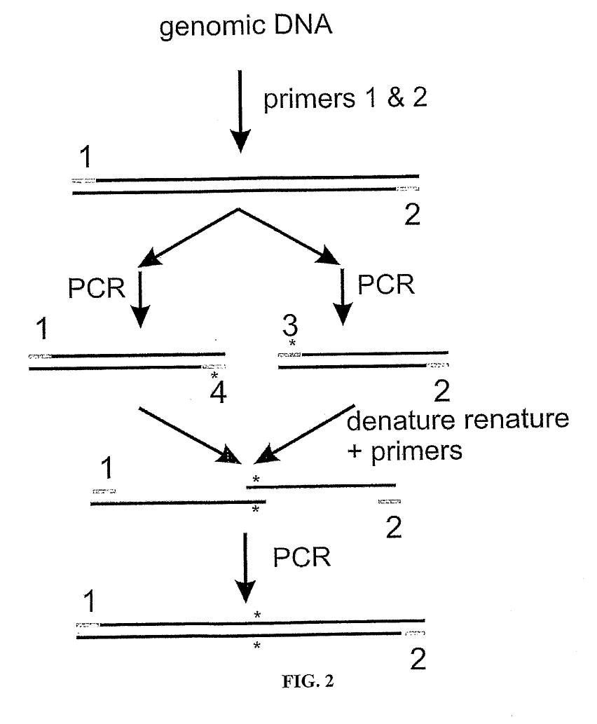

New mutations (FIG. 2) are introduced in the following manner by PCR based synthesis using mutated oligonucleotides as primers to introduce the desired mutations:

Two mutations are made were based on the data obtained with camelid V_{HH} regions, in particular, a phenyalanine (F) at position 37 and a glutamic acid (E) at position 44.

Leucine 45 is substituted with a glutamine (Q. i.e. different from camelid), because it establishes a hydrogen bridge with the carbonyl group of glycine 116, establishing a structure similar to that observed in camelid V_{HH} around residue 116 and provides stacking. In addition, a glycine (G) substitutes the tryptophan (W) or tyrosine (Y) at position 47 to establish an interaction with the tyrosine (Y) at position 58.

These 4 amino acid changes are introduced in all of the human V gene sequences shown in FIG. 2 (top).

In addition, two other substitutions are introduced either together in the V gene sequences 3-11, 3-23 and 3-53 or individually in the 3-23 VH sequence only. At position 5, a valine (V) or leucine (L) is replaced by a glutamine (Q) to increase the surface hydrophilicity of the V_{H} region. The proline (P) at position 14 is substituted with an alanine (A) to establish an interaction with amino acid 127 in the DJ region and reinforce the structure of the V_{H} domain. Such interaction does not take place with the normal P at position 14.

### Mutagenesis of V Gene Segments Primarily Using VH3-23 as the Example

A routine overlapping PCR strategy was used to introduce the desired amino acid changes into the human V_{H} domains (FIG. 2). In the example below, the 6 amino acid changes through mutations were introduced in two overlapping PCR steps. The V gene segments were isolated by PCR from genomic human DNA using primers 1 and 2 to provide the starting material for mutagenesis.

### Steps:

Primers 3 and 4 contained all the base changes for the amino acid changes 37I or 37V to F, G44 to E, L45 to Q and W47 to G. Primer 1 and primer 4 were used to modify the 5′ half of the V gene segment while primers 2 and 3 were used to modify the 3′ half. Primers 3 and 4 are different for different V gene segments, e.g. VH3-11 has an isoleucine I (DNA sequence ATC) whereas VH3-23 has a valine V (DNA sequence GTC), necessitating different primers 3 and 4. The primers are removed by chromatography (Qiagen kit).

The resulting fragments containing the mutations are mixed, denatured, renatured and PCR amplified. The resulting long product has the desired nucleotide changes for the conversion of the 4 targeted amino acids to F, E, Q and G.

This fragment was used for the introduction of further mutations using primers 5 and 6. These contained the DNA base changes required for the conversion of leucine (L) or valine (V) at position 5 to glutamine (Q) and proline (P) to alanine (A) at position 14. Primers 1 and 6 were used to obtain the 5′ half. Primers 2 and 5 were used to obtain the 3′half. The primers were removed by chromatography (Qiagen kit).

The two halves were combined in one PCR reaction as described in step 2. The resulting long fragments had the desired Q and A codon changes in addition to the changes described above.

The mutated V gene segments were digested with SalI at the 5′ end and XhoI at the 3′ end and cloned into Bluescript for sequence analysis.

The desired changes were confirmed by sequence analysis. The sequences of the primers used were:

Sequence of VH3-23 around amino acid position 50. The top line shows the mutated sequence, where the changed residues are underlined, and the bottom line shows the starting sequence.

TCTGGATTCACCTTTAGCAGCTATGCCATGAGCTGGTTCCGCCAGGCTCCAGGGAAGGAG (SEQ ID NO: 1)
TCTGGATTCACCTTTAGCAGCTATGCCATGAGCTGGGTCCGCCAGGCTCCAGGGAAGGGG (SEQ ID NO: 2)
CAGGAGGGGGTCTCAGCTATTAGTGGTAGTGGTGGTAGCACATACTACGCAGACTCCGTG (SEQ ID NO: 1)
CTGGAGTGGGTCTCAGCTATTAGTGGTAGTGGTGGTAGCACATACTACGCAGACTCCGTG (SEQ ID NO: 2)

Forward VH3-23/FEQG primer 3

(SEQ ID NO: 3)

5′ CTGGTTCCGCCAGGCTCCAGGGAAGGAGCAGGAGGGGGTC

Reverse VH3-23/FEQG primer 4

(SEQ ID NO: 4)

5′ GACCCCCTCCTGCTCCTTCCCTGGAGCCTGGCGGTACCAG

Sequence of VH3-23 around amino acid position 10. The top line shows the mutated sequence, where the changed residues are underlined and the bottom line shows the starting sequence.

AGTTTCTGACCAGGGTTTCTTTTTGTTTGCAGGTGTCCAGTGTGAGGTGCAGCTGCAGGA (SEQ ID NO: 5)
AGTTTCTGACCAGGGTTTCTTTTTGTTTGCAGGTGTCCAGTGTGAGGTGCAGCTGTTGGA (SEQ ID NO: 6)
GTCTGGGGGAGGCTTGGTACAGGCTGGGGGGTCCCTGAGACTCTCCTGTGCAGCCTCTGG (SEQ ID NO: 5)
GTCTGGGGGAGGCTTGGTACAGCCTGGGGGGTCCCTGAGACTCTCCTGTGCAGCCTCTGG (SEQ ID NO: 6)

Forward VH3-23/5+14 primer 5

(SEQ ID NO: 7)

5′ CAGCTGCAGGAGTCTGGGGGAGGCTTGGTACAGGCTGGG

Reverse VH3-23/5+14 primer 6

(SEQ ID NO: 8)

5′ CCCAGCCTGTACCAAGCCTCCCCCAGACTCCTGCAGCTG

Forward IGHV3-23F primer 1

(SEQ ID NO: 9)

5′ GTGGTCGACGATGGAAAGATAGATACCAACATG

Reverse IGHV3-23R primer 2

(SEQ ID NO: 10)

5′ GTGCTCGAGCATCTCTGTAAGCGTCAATCTGC

The primers to achieve the amino acid changes at positions 5 and 14 were also synthesized separately using the template that already had the FEQG changes (after step 2 above).

For the change at position 5, the primers 5 and 6 in the figure had a different sequence. For L>Q at position 5:

Forward VH3-23/5L-Q primer 5

5′ GGTGCAGCTGCAGGAGTCTGG (SEQ ID NO: 11)

Reverse VH3-23/5L-Q primer 6

5′ CCAGACTCCTGCAGCTGCACC (SEQ ID NO: 12)

For P>A at position 14

Forward VH3-23/14P-A primer 5

5′ CTTGGTACAGGCTGGGGGGTC (SEQ ID NO: 13)

Reverse VH3-23/14P-A primer 6

5′ GACCCCCCAGCCTGTACCAAG (SEQ ID NO: 14)

Different primers 5 and 6 were used for the different V gene segments that had a different sequence in the relevant areas.

All the desired changes for the different V gene segments have been cloned and sequenced.

The next phase of the construction is the insertion of the V gene segments into the human heavy chain locus. This consists of several steps:

1. This locus is in principle the same as that described in [16] with two differences. The locus does not contain lox or frt sites and instead of the llama V_{HH} regions it contains a PspI meganuclease site. These loci have been constructed successfully.
2. A separate BAC construct was made that contains a single XhoI site flanked on either side by a PspI meganuclease site.
3. Each modified V gene segment is removed from Bluescript by SalI and XhoI digestion and cloned into the XhoI site of the modified BAC3.6. This restores the XhoI site at the 3′ end of the V gene segment but destroys the SalI at the 5′ end of the V gene segment.
4. The resulting construct is cut at its unique XhoI site and the next V gene segment is cloned into the site, again leaving a unique XhoI at the 3′ end of now a V gene segment dimer.
5. This cycle is repeated until the desired multimer of V gene segments is obtained.
6. The multimer is removed from the BAC3.6 and cloned into the unique PspI site of the locus described in step 1.
7. The resulting completely human heavy chain-only locus is microinjected into fertilized eggs to obtain transgenic mice for immunization.

The entire locus is digested from the PAC by a NotI digest [16], purified by routine procedures and injected into fertilized mouse eggs by standard procedures to obtain transgenic mice carrying the locus as part of its genome [16]. It is not necessary to use mouse strains in which endogenous murine immunoglobulin genes have been deleted or expression repressed, since allelic exclusion determines which locus (endogenous or transgene) will result in a productive expression. Our preferred strategy is to cross mice carrying functional transgenes into a background with minimized or no endogenous gene expression and to use the progeny derived from these crosses for the generation of heavy chain-only antibodies against target antigens. This particular locus was injected in both types. The results shown below are from injections in wt fertilized eggs.

An alternative and more laborious approach for the generation of human heavy chain-only antibody is to use homologous recombination strategies to replace the non-human mammalian host's (in this instance mouse) V, D J and segments with human natural and/or engineered V segments, human D and J segments and to replace the constant regions with human heavy chain constant regions devoid of C_{H}1. If only human V_{H} domains are required, then host constant region gene segments devoid of C_{H}1 would suffice.

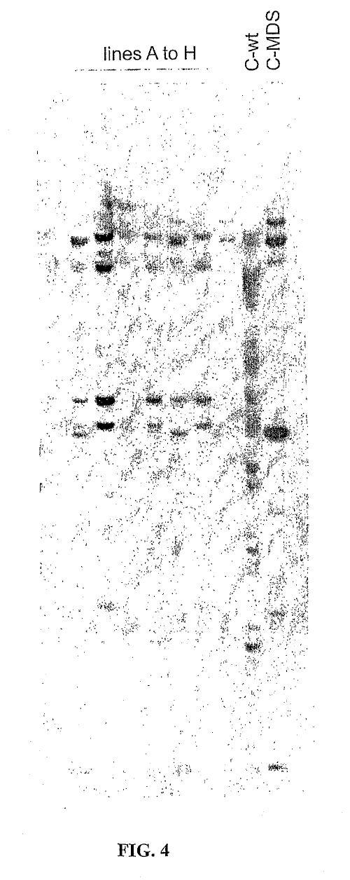

Using established oocyte injection technology, the mice are shown to be transgenic by standard procedures such as routine PCR analysis of the different regions or by routine Southern blotting that detect the different regions of the transgenic locus (FIG. 4).

If the introduced human heavy chain loci are functional and soluble antibody expressed, we would expect the transgenes to respond as part of the host response to its natural environment. Human heavy chain-only antibody would be present in plasma and human heavy chain-only antibody mRNA would be present in circulating B-cells. Sequence analysis of cloned mRNA will identify (i) preferred introduced mutations, and (ii) any somatic mutations resulting in the presence of stable and soluble circulating human heavy chain-only antibody.

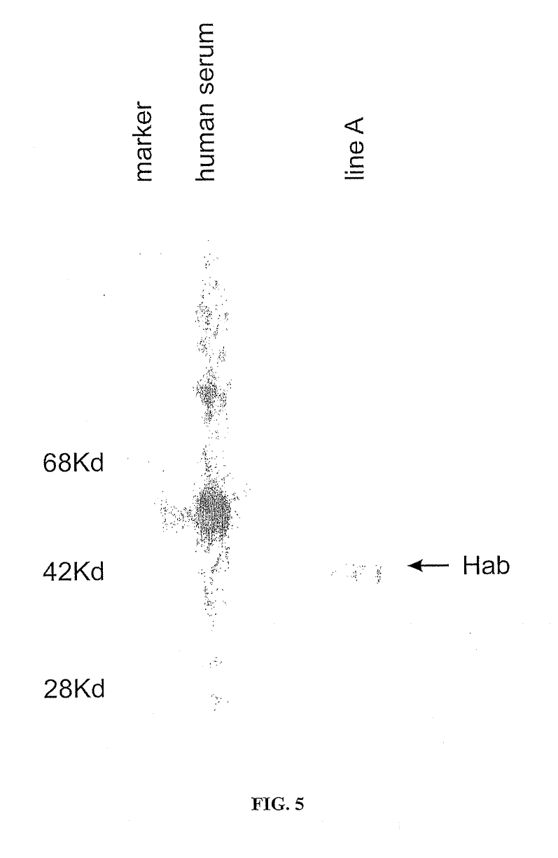

To show that human heavy chain-only antibodies and that human heavy chain mRNA is produced from the transgenes, blood is taken and serum and white cells recovered. Western blotting using an antibody that specifically detects human IgG (Sigma goat anti-human IgG coupled to peroxidase, FIG. 5) demonstrates that human heavy chain-only antibody is present in plasma.

RT-PCR of the RNA prepared from the cells in the same blood sample using the following primers shows the presence of the expected mRNA transcripts.

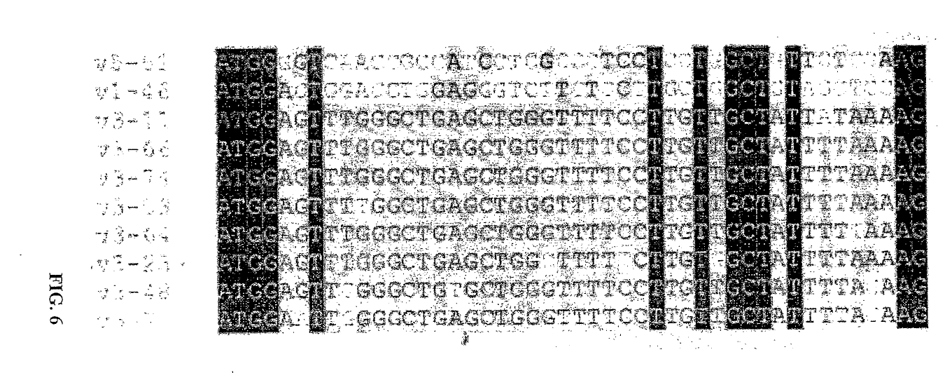

The RNA was reverse transcribed (oligodT) and made into cDNA, using the forward primers derived from the ATG start codon region of the different V gene segments illustrated below (FIG. 6).

Four primers were synthesized:

VH3all GGCTGAGCTGGGTTTTCCTTGTTGCTATT (SEQ ID NO: 15) which will
synthesize V3-11, 66, 74, 53, 64, 48;
VH3-51 CCGCCATCCTCGCCCTCCTCCTGGCTGTT (SEQ ID NO: 16) recognizing
V5-51;
VH3-46 CCTGGAGGGTCTTCTGCTTGCTGGCTGTA (SEQ ID NO: 17) recognizing
V1-46; and
VH3-23 GGCTGAGCTGGCTTTTTCTTGTGGCTATT (SEQ ID NO: 18) recognizing
V3-23.

The resulting cDNA was subsequently PCR amplified using the same forward primers and the reverse primers that are identical as those described in [16] specific for Cγ2 and Cγ3 constant regions.

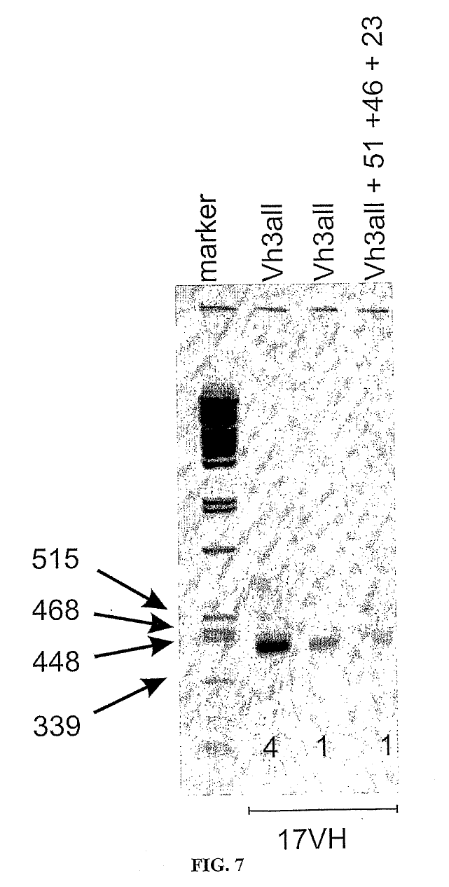

PCR amplification of the different samples using a combination of forward and reverse primers shows that the appropriate size fragment (approximately 390 bp) is produced with the different sets of primers (FIG. 7). The polymerase in the PCR reaction was PFU polymerase (proof reading polymerase to prevent mutations) followed by an addition of A (i.e. not another round of PCR) using a non-proof reading polymerase (to allow cloning into PGMTeasy).

Importantly, it is clear that the fragment is somewhat diffuse, indicating that it contains different PCR products of slightly different length, as would be expected from the process of VDJ recombination and mutation. Thus, the locus is expressed and results in antibody production.

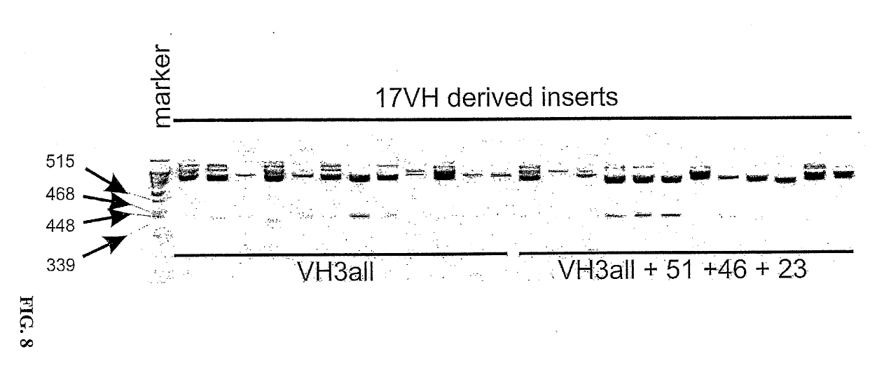

In order to identify preferred introduced mutations derived from V gene segments, and to identify further mutations present in the V_{H} domain due to affinity maturation and selection by the mouse, the PCR products (FIG. 7) were cloned by standard AT cloning in pGEMTeasy and DNA prepared by standard methods. These DNA samples were cleaved with EcoRI and analysed by gel electrophoresis. This shows that they have different size inserts (FIG. 8), hence different recombinations/mutations are made in the mouse.

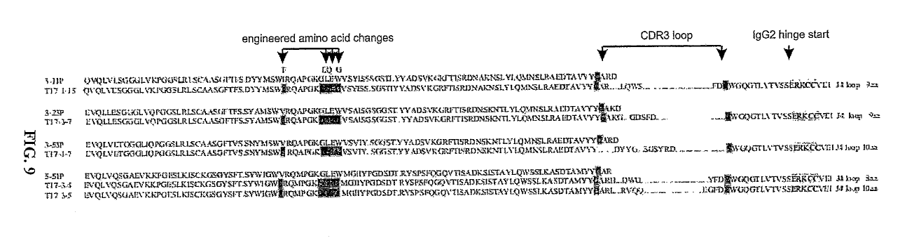

Plasmids with single inserts are subsequently sequenced. This indeed confirms that different recombinations and mutations are generated by the mouse immune system (FIG. 9) using human V gene segments engineered to enhance solubility and stability. The presence of circulating heavy chain-only antibody in plasma under physiological conditions indicates that transgene-derived soluble and stable human heavy chain-only antibody is synthesised and secreted by B-cells. Cloned and expressed human V_{H} domains provide a source of protein for comparative physical studies to identify somatic mutation(s) introduced in vivo which impart improved solubility and stability relative to V_{H} domains lacking these mutations.

### Example 3

In example 2 we teach: the prediction of new mutations to confer improved solubility and stability into V_{H} domains; the introduction of engineered V gene segments into a heavy chain locus; the analysis of transgene expression; the identification of preferred mutated V gene segments present in VH domains as a result of VDJ rearrangement leading to the presence of soluble and stable heavy chain-only antibody circulating under physiological conditions in plasma.

In a third example, a number of further preferred mutations deduced in silico (see example 1) are introduced into human V gene segments (for examples of sequences see FIG. 1). In this example, the mutations are such that a charged amino acid is created at position 45 in analogy with the presence of a charged amino acid at that position in camelid V_{HH} regions so as, to improve solubility, and further synergistic mutations are introduced elsewhere so as to maintain V_{H} stability.

Thus (see FIG. 3 for the sequences and the public databases PDB):
L is changed to R (or another charged amino acid or C) at position 45 with a change to E or S or A (E>S>A) at position 44 which is favourable to stabilize the loop position of position 45 with π stacking with Y at position 95 and W at position 118;
a change to R at position 52 which would stabilize through an interaction with the main chain of V_{H}. This would be favourable independent of other mutations.

A change of R at position 45 is also favourable combined with a change at position 61 to K. This mutation would also be favourable independent of 45.

Changes at 37 (to F) and 5 (to Q) would still be favourable as described in example 1. The GHG loop in the llama sequence starting at position 25 and ending at 38 (35 in human V_{H}) would be favourable if transplanted to the human V_{H} in combination with an R (or S) at position 27 as it increases hydrophilicity and would provide stabilization if a contact would be made with position 77 by changing it to a T.

Engineered human V gene segments (independent of subclass) can then be introduced into a human heavy chain only locus, expressed as a transgene, and preferred engineered mutations and any additional preferred mutations resulting from affinity maturation identified as described in example 2 above.

Further transgenes can be generated based on the principles described above which combine mutations at the V_{H}/V_{L} interface, so improving solubility, whilst additional distal mutations based on the in silico analysis of the introduced mutation at the V_{H}/V_{L} interface are introduced to maintain V_{H} maintain stability. Selection and natural maturation processes in vivo result in secretion into serum of heavy chain-only antibodies which are soluble and stable under physiological conditions.

### REFERENCES

[1] Kabat, E., Wu, T. T., Perry, H. M., Gottesman, K. S., and Foeller, C. (1991) United States Public Health Services Publication No. 91-3242, National Institutes of Health, Bethesda, Md.
[2] Jaton et al., Recovery of antibody activity upon reoxidation of completely reduced polyalanyl heavy chain and its Fd fragment derived from anti-2,4-dinitrophenyl antibody. (1968) Biochemistry, 7, 4185-4195
[3]] Xu J L, Davis M M. Diversity in the CDR3 region of V(H) is sufficient for most antibody specificities. Immunity 13, 37-45 (2000)
[4] Hendershot et al., Assembly and secretion of heavy chains that do not associate posttranslationally with immunoglobulin heavy chain-binding protein (1987) J. Cell Biol., 104, 761-767;
[5] Brandt et al., Loss of a consensus splice signal in a mutant immunoglobulin gene eliminates the CH1 domain exon from the mRNA. (1984) Mol. Cell. Biol., 4, 1270-1277
[6] Hamers-Casterman et al., Naturally occurring antibodies devoid of light chains. (1993) Nature, 363, 446-448
[7] Stanfield et al., Crystal structure of a shark single-domain antibody V region in complex with lysozyme. (2004) Science, 305, 1770-1773
[8] Desmyter et al., Crystal structure of a camel single-domain VH antibody fragment in complex with lysozyme. (1996) Nat. Struct. Biol., 3, 803-811
[9] Riechmann, L. & Muyldermans, S. Single domain antibodies: comparison of camel VH and camelised human VH domains. J Immunol Methods 231, 25-38 (1999).
[10] Ward et al., Binding activities of a repertoire of single immunoglobulin variable domains secreted from Escherichia coli. (1989) Nature, 341, 544-546
[11] De Genst E, Silence K, Ghahroudi M, Decanniere K, Loris R, Kinne J, Wyns L & Muyldermans S. Strong in vivo maturation compensates for structurally restricted H3 loops in antibody repertoires. J Biol. Chem. 280, 14114-21 (2005)
[12] Davies, J and Riechmann L. Biotechnology (1995) vol 13, 475-479 Antibody VH domains as small recognition units
[13] de Genst et al., Antibody repertoire development in camelids. Dev Comp Immunol. 2006; 30:187-98
[14]] Davies, J and Riechmann, L. Single antibody domains as small recognition units: design and in vitro antigen selection of camelized, human VH domains with improved protein stability. Protein Eng. 9, 531-7 (1996).
[15] Lutz and Muyldermans, Single domain antibodies: comparison of camel VH and camelised human VH domains. (1999) J. Immuno. Methods, 231, 25-38
[16] Rick Janssens, Sylvia Dekker, Rudi W. Hendriks, George Panayotou, Alexandra van Remoortere, John Kong-a San, Frank Grosveld and Dubravka Drabek, Generation of heavy chain only antibodies in mice, Proc. Natl. Acad USA 2006, Oct. 10; 103:15130-5.
[17] Guglielmi L, Le Bert M, Comte I, Dessain M L, Drouet M, Ayer-Le Lievre C, Cogne M, Denizot Y Combination of 3′ and 5′ IgH regulatory elements mimics the B-specific endogenous expression pattern of IgH genes from pro-B cells to mature B cells in a transgenic mouse model. Biochim Biophys Acta. 2003 Oct. 21; 1642(3):181-90.
[18] Spinelli S, Frenken L, Bourgeois D, de Ron L, Bos W, Verrips T, Anguille C, Cambillau C, Tegoni M. The crystal structure of a llama heavy chain variable domain. Nat Struct Biol. 1996 September; 3(9):752-7.

1. A method for producing a heavy chain-only antibody comprising:
challenging with an antigen a non-human mammal having a heavy chain locus which:
comprises a plurality of V gene segments which encode one or more amino acid mutations (i) at the V_{L} interface so as to reduce hydrophobicity and (ii) at other positions so as to overcome structural instability;
comprises at least one D gene segment and at least one J gene segment;
lacks gene segments encoding a CH1 domain or has been engineered to prevent expression of a functional CH1 domain; and
when expressed in response to antigen challenge, produces a heavy chain-only antibody devoid of CH1, having a soluble VH domain encoded by a VH gene which includes a preferred V gene segment incorporated as a result of VDJ rearrangement, and affinity maturation into said VH gene.

2. The method of claim 1, wherein the preferred V gene segment and optionally the preferred D and J segments are further modified by affinity maturation resulting in enhanced stability and solubility of the resulting V_{H} domain incorporated into the heavy chain-only antibody

3. The method of claim 1 or claim 2 wherein said non-human mammal is produced by:
producing in vitro a transgene including said locus; and
introducing said transgene into a suitable cell from which said non-human mammal is to be produced.

4. The method of claim 3, wherein the cell is an embryonic stem cell or an oocyte.

5. The method of claim 1 or claim 2, wherein said non-human mammal is produced by homologous recombination in which said plurality of mutated V gene segments, and, optionally, said D and J gene segments, replace the equivalent gene segments in an endogenous heavy chain locus, said heavy chain loci lacking CH1 functionality in the non-human mammal.

6. The method claim 1 or 2, wherein the non-human mammal includes multiple heavy chain loci at least one of which is as defined in claim 1 and each of which is on a different chromosome.

7. The method of claim 6, wherein one or more loci comprise natural V gene segments and the remainder comprise one or more engineered V gene segments.

8. The method of claim 6, wherein the loci on each chromosome are different

9. The method of claim 6, wherein the loci on each chromosome are the same.

10. The method of claim 1 or 2, wherein the V gene segments are mutated human V gene segments.

11. The method of claim 1 or 2, wherein the locus includes multiple D gene segments.

12. The method of claim 1 or 2, wherein the locus includes multiple J gene segments.

13. The method of claim 1 or 2, wherein the locus contains at least one gene segment encoding a constant region.

14. The method of claim 13, where in the locus contains multiple gene segments encoding constant regions.

15. The method of claim 13, wherein the gene segments encoding a constant region are human.

16. The method of claim 1 or 2, wherein each D and J gene segment is human.

17. The method of claim 1 or 2, wherein each V gene segment encodes a protein which has a mutation at one or more of positions 37, 44, 45 and 47.

18. The method of claim 17, wherein each V gene segment encodes a protein which has mutations at all of positions 37, 44, 45 and 47.

19. The method of claim 17, wherein the V gene segment encodes a protein wherein at position 37, the residue is phenylalanine, at position 44, the residue is glutamic acid, at position 45, the residue is glutamine and/or at position 47, the residue is glycine.

20. The method of claim 1 or 2, wherein each V gene segment encodes a protein which has a mutation at either of positions 5 and 14.

21. The method of claim 20, wherein each V gene segment encodes a protein which has mutations at both of positions 5 and 14.

22. The method of claim 20, wherein the V gene segment encodes a protein wherein at position 5, the residue is glutamine and/or at position 14, the residue is alanine.

23. A heavy chain-only antibody having a VH domain encoded in part by any one of the V gene segments set forth in FIG. 3.
